(19) United States
(12) Patent Application Publication
(10) Pub. No.: US 2018/0197435 A1
(43) Pub. Date: Jul. 12, 2018

(54) HANDCUFFING TRAINING APPARATUS

(71) Applicant: BLUE TRAINING SYSTEMS INC., Vancouver (CA)

(72) Inventor: Clive Milligan, Delta (CA)

(21) Appl. No.: 15/864,422

(22) Filed: Jan. 8, 2018

Related U.S. Application Data
(60) Provisional application No. 62/444,626, filed on Jan. 10, 2017.

(51) Int. Cl. G09B 19/00; G09B 5/04

(52) U.S. Cl. CPC G09B 19/003; A61F 13/107; G09B 5/04

(57) ABSTRACT

The present disclosure relates to a handcuff training apparatus, a method of using thereof, and a kit comprising the handcuffing training apparatus. The handcuff training apparatus comprises instructions provided thereon for directing and instructing a first individual on how to apply handcuffs onto a second individual and/or guiding lines provided thereon for guiding the first individual on where to apply the handcuffs around the second individual's wrists. The handcuff training apparatus can be made of a suitable material such as, but not limited to, an adhesive tape, a flexible bi-stable spring band, or an elastomeric band wherein the elastomeric band wraps and couples around the second individual's wrist by elastomeric tension.

### CROSS-REFERENCE TO RELATED APPLICATION

This application claims priority to U.S. Provisional Application No. 62/444,626, filed Jan. 10, 2017.

### TECHNICAL FIELD

The present disclosure relates to a handcuffing training apparatus, a method of using thereof, and a kit comprising the handcuffing training apparatus.

### BACKGROUND

Law enforcement officers are often called upon to restrain individuals (e.g. suspects, criminals, individuals with self-destructive behaviour, etc. . . . ) in a manner that is effective, is efficient, and minimizes harm to the restrained individuals. Such acts of restraint may be aided by the use of a restraining device such as handcuffs.

There are various techniques of applying handcuffs onto an individual. In a non-limiting example of basic handcuffing procedures, the hands of the individual are preferably positioned behind the individual's back with the palms facing outward (commonly known as back-to-back). Handcuffs are applied around the individual's wrists. If possible and tactically feasible, handcuffs should be double-locked. In a non-limiting example of speed handcuffing (“speed-cuffing”) procedure, handcuffs are drawn by its chain, and the handcuffs are first pressed onto one of the restrained individual's wrists and then immediately onto the other wrist of the restrained individual. The handcuffs are then adjusted to firmly secure the handcuffs around the wrists of the restrained individual. The wrist is generally understood to be defined by the carpal bones, including the joint (radiocarpal joint) between the radial bone of the forearm and the carpal bones. The carpal bones are made up of eight small bones that collectively connect the forearm to the hand.

During the application of handcuffs to a subject, an enforcement agent (e.g an officer, sheriff, soldier, guard, or the like) should use best practice principles to minimize injury to himself/herself and the subject. Firstly, handcuffs should be loaded in a properly prescribed fashion in the agent's handcuff pouch/holder. For example, the teeth on the single bar of the handcuff should be aligned so that only one tooth or two teeth maintain pressure on the ratchet, thereby allowing for the cuffs to be secured smoothly onto the subject's wrists and reducing the likelihood of injury to the subject. Such alignment also decreases the time required to apply the handcuffs onto the subject, thereby allowing the agent to exercise restraint of the subject in a shorter time. Secondly, the subject should be physically controlled first before the handcuffs are applied onto the subject. Holding onto the handcuffs prior to having control of the subject decreases the agent's ability to use both hands to manipulate the subject into a position of advantage for the agent and therefore should be avoided. Thirdly, the agent should apply the handcuffs onto the wrist by pressing/pushing the handcuffs onto the wrist and pressing/pushing the wrist into the cuff. Such action prevents actual striking of the metal (or other materials) onto the bones of the subject's hands/arm, thereby reducing risk of injury to the subject. Fourthly, it is important to check for tightness of the handcuffs around the subject's wrists and to ensure that the handcuffs are securely fastened around the subject's wrists. This is commonly done by inserting the tip of a finger into the area space between the handcuff and the subject's wrist, if tactically feasible. Fifthly, it is important to ascertain the comfort level of the subject. Metal cuffs are not designed to be comfortable, but checking the comfort level of the restrained subject may build rapport, and potentially de-escalate a situation; more importantly, and if needed, adjustments may be made. An agent may then be able to note the subject's verbal or non-verbal responses in a notebook. Sixthly, it is important to ensure that the handcuffs are double locked. Handcuffs may be double locked by activating a small metal pin that occludes the inner ratchet mechanism. Double locking prevents over-tightening of the handcuffs and also makes it more difficult for the subject to escape from the handcuffs.

Notwithstanding the foregoing non-limiting examples of best practice principles, and particularly during a physical or violent struggle, an agent (e.g. law enforcement officer) may apply handcuffs on a subject's wrists with a less-than-ideal technique. Such less-than-ideal technique may include (but is not limited to) applying the handcuffs to the wrong place around the wrists of the restrained individual, using excessive force in applying the handcuffs around the wrists of the restrained individual, and/or over-tightening of handcuffs around the wrists of the restrained individual. Such less-than-ideal techniques can cause trauma and damage to the wrists including, but not limited to, severe bruising, lacerations, bone fracture, nerve damage, and lesions (Haddad et al., Complaints of pain after use of handcuffs should not be dismissed, BMJ, 1999, 318: 55).

Appropriate handcuff application training may lessen the occurrence of such trauma and damage to the wrists during the application of handcuffs around the wrists of the restrained individual. Law enforcement officers, such as but not limited to police officers, routinely practice correct handcuffing application in training exercises in order that the correct application be used when on duty. During these training exercises, police officers will practice handcuffing application on each other and wrists can become bruised if they are not protected and the correct handcuffing procedure is not followed.

### SUMMARY

The present disclosure relates to a handcuffing training apparatus, a method of using thereof, and a kit comprising the handcuffing training apparatus.

According to an aspect of the disclosure, there is a handcuff training apparatus comprising a first surface and a second surface that is opposite the first surface, the first surface comprising one or more instructions provided thereon and/or one or more guiding lines extending along a length of the handcuff training apparatus, the handcuff training apparatus adapted to circumscribe and removably couple to a wrist of an individual.

The handcuff training apparatus may stretch in a longitudinal direction.

The handcuff training apparatus may be made of kinesiology tape.

The handcuff training apparatus may be for use in training a first individual on how to apply handcuffs onto a second individual in a manner that minimizes discomfort for the second individual.

According to another aspect of the disclosure, there is a method of using a handcuff training apparatus, the method comprising: (a) providing the handcuff training apparatus; (b) wrapping the handcuff training apparatus around a wrist of an individual; and (c) applying a handcuff onto the wrist of the individual by (i) following the one or more instructions provided on the surface of the handcuff training apparatus, and/or (ii) using the guiding lines extending along the length of the handcuff training apparatus as a reference.

According to another aspect of the disclosure, there is a handcuff training kit comprising: (i) a handcuff training apparatus; and (ii) instructions on how to use the handcuff training apparatus.

The handcuffing training apparatus may prevent injury to wrists from potential impact and abrasions of handcuffs applied in training or in real-life situations. The handcuffing training apparatus may provide a protective barrier against pathogens brought into a training environment or present on existing handcuffs. The handcuffing training apparatus may actively remind a trainee of best practice principles associated with applying handcuffs onto a subject.

The handcuffing training apparatus, when provided in the form of a suitable tape such as a kinesiology tape, may increase circulation to tissues around the wrist, and may prevent injuries caused by hyper-flexion/extension of the wrist/hand of the subject.

This summary does not necessarily describe the entire scope of all aspects of the disclosure. Other aspects, features and advantages will be apparent to those of ordinary skill in the art upon review of the following description of specific embodiments.

### BRIEF DESCRIPTION OF THE DRAWINGS

In the accompanying drawings, which illustrate one or more exemplary embodiments:

FIG. 1(a) is a top plan view of a handcuff training apparatus according to an embodiment.

FIG. 1(b) is a side view of the handcuff training apparatus of FIG. 1(a).

FIG. 2 is a top plan view of a handcuff training apparatus according to another embodiment.

FIG. 3 is a top plan view of a handcuff training apparatus according to yet another embodiment.

### DETAILED DESCRIPTION

Directional terms such as “top”, “bottom”, “upwards”, “downwards”, “vertically”, and “laterally” are used in the following description for the purpose of providing relative reference only, and are not intended to suggest any limitations on how any article is to be positioned during use, or to be mounted in an assembly or relative to an environment. Any element expressed in the singular form also encompasses its plural form. Any element expressed in the plural form also encompasses its singular form. The use of the word “a” or “an” when used herein in conjunction with the term “comprising” may mean “one”, but it is also consistent with the meaning of “one or more”, “at least one”, and “one or more than one”.

As used herein, the terms “comprising”, “having”, “including”, and “containing”, and grammatical variations thereof, are inclusive or open-ended and do not exclude additional, un-recited elements and/or method steps. The term “consisting essentially of” when used herein in connection with a composition, use or method, denotes that additional elements, method steps or both additional elements and method steps may be present, but that these additions do not materially affect the manner in which the recited composition, method or use functions. The term “consisting of” when used herein in connection with a composition, use or method, excludes the presence of additional elements and/or method steps.

The use of the word “about”, when used in reference to a recited numerical value or a numerical value that may be inferred, means plus or minus 10% of the recited numerical value or a numerical value that may be inferred.

The present disclosure relates to a handcuff training apparatus. The handcuff training apparatus may comprise instructions provided thereon for directing or prompting a first individual (e.g. law enforcement officer, security officer etc.) on how to engage a second individual (e.g. another law enforcement officer or an actor portraying a suspect, violent criminal, or person in mental distress), how to apply handcuffs onto the second individual, and what to do after the handcuffs have been applied onto the second individual. The handcuff training apparatus may additionally or alternatively comprise guiding lines also provided thereon for guiding the first individual on where to apply the handcuffs around the second individual's wrists. The handcuff training apparatus can be made of a suitable material such as, but not limited to, an exercise kinesiology tape, an adhesive tape, a flexible bi-stable spring band (e.g. a slap bracelet) sealed with a suitable material such as, but not limited to, fabric, silicone, or plastic, or an elastomeric band wherein the elastomeric band wraps and couples around the second individual's wrist by elastomeric tension.

The present disclosure also relates to a method of using the handcuff training apparatus.

The present disclosure also relates to a kit comprising the handcuff training apparatus and instructions on how to use the handcuff training apparatus.

### Handcuff Training Apparatus

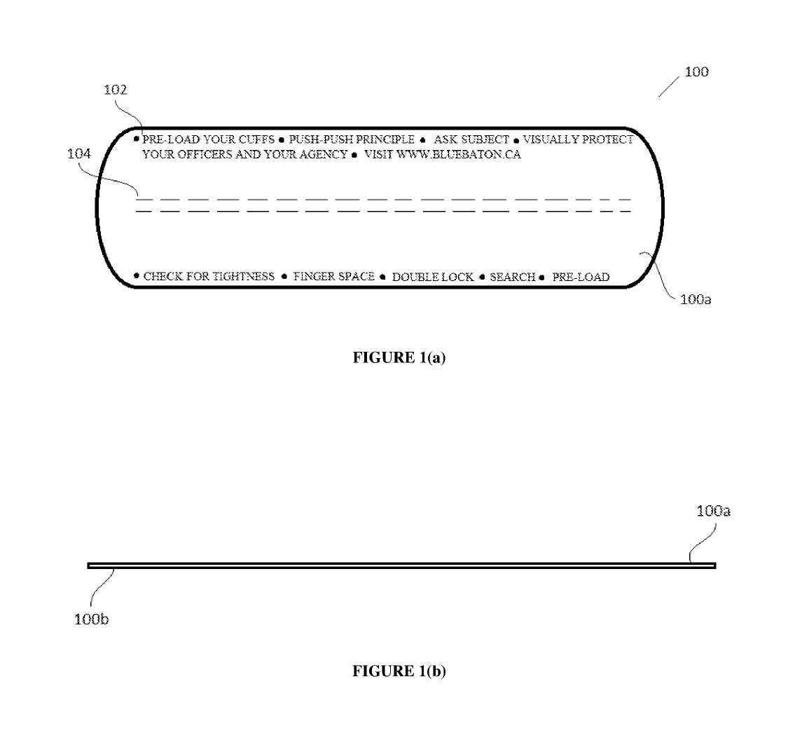

Referring to FIG. 1, and according to an embodiment of the disclosure, there is shown a handcuff training apparatus 100 adapted for training a first individual on how to apply handcuffs onto the wrists of a second individual. The handcuff training apparatus 100 comprises a top surface 100a and a bottom surface 100b that is opposite the top surface 100a. The handcuff training apparatus 100 also comprises instructions 102 and guiding lines 104 provided on the top surface 100a. The handcuff training apparatus optionally comprises a protective layer (not shown) overlaying the top surface 100a to protect the instructions 102 and guiding lines 104 thereon.

Instructions 102 are provided on the top surface of 100a, and are permanently imprinted onto the top surface 100a. Non-limiting examples of instructions include “pre-load your cuffs”, “push & push principle”, “ask subject”, “visually protect your officers and your agency”, “check for tightness”, “finger space”, “double lock”, and “search”. The instructions 102 direct or prompt the first individual (e.g. law enforcement officer, security officer etc.) on how to engage a second individual (e.g. a law enforcement officer or actor portraying a suspect, violent criminal, or person in mental distress), how to apply handcuffs onto the second individual, and what to do after the handcuffs have been applied onto the second individual. Such instructions are preferably listed in sequential order or steps, though the provided instructions can be randomized. As contemplated in this embodiment, instructions 102 are provided in sequential order or steps.

As contemplated in this embodiment, instructions 102 are provided along the length of top surface 100a, and in a sequence of steps, from left to right, along the length of the handcuff training apparatus 100. In other embodiments, instructions can be listed in sequential order down the width of the handcuff training apparatus 100, or in any suitable manner over the top surface 100a.

Guiding lines 104 provide guidance to the first individual on where to contact the handcuffs against the handcuff training apparatus 100 (and therefore the second individual's wrist). The guiding lines 104 extend along or about the centre-line of the top surface 100a of the handcuff training apparatus 100. For example, the guiding lines 104 can be about 10%, 9.5%, 9%, 8.5%, 8%, 7.5%, 7%, 6.5%, 6%, 5.5%, 5%, 4.5%, 4%, 3.5%, 3%, 2.5%, 2%, 1.5%, 1%, 0.5%, or 0% off-centred. Guiding lines may have different visual arrangements. For example, the guiding lines can be, but is not limited to, a single solid line, a single stippled line, double solid lines, double stippled lines, single solid line in combination with a single stippled line, triple solid lines, triple stippled lines, and the like. As contemplated in this embodiment, the guiding lines 104 are double stippled lines.

As contemplated in this embodiment, instructions 102 are provided on both side of the guiding lines 104, in the direction of the length of the top surface 100a and along the edges of the top surface 100a. In other embodiments, instructions can be provided on only one side of the guiding lines in the direction of the length of top surface of the handcuff training apparatus, or in any suitable spatial and visual arrangement relative to the guiding lines. In other embodiments, instructions can be listed in sequential order down the width of the handcuff training apparatus, and guiding lines can be printed over at least part of the instructions. In other embodiments, the instructions can be provided over at least a part of the guiding lines.

The bottom surface 100b is adapted to contact and circumscribe the wrist of the second individual. As contemplated in this embodiment, the bottom surface 100b comprises an adhesive substance. The adhesive substance can be on part of or all of bottom surface 100b. The adhesive substance is adapted to removably couple the handcuff training apparatus 100 to and around the second individual's wrist. In other embodiments, the bottom surface 100b does not comprise an adhesive substance.

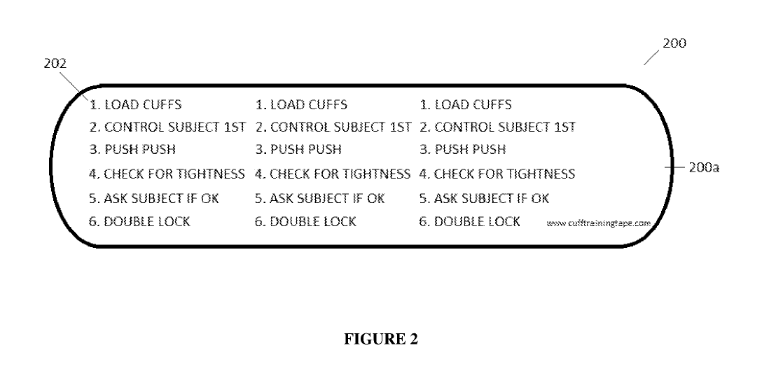

Referring to FIG. 2, and according to another embodiment of the disclosure, there is a handcuff training apparatus 200 adapted for training a first individual on how to apply handcuffs onto the wrists of a second individual. The handcuff training apparatus 200 comprises a top surface 200a and a bottom surface (not shown) that is opposite the top surface 200a. The handcuff training apparatus 200 further comprises instructions 202 provided on the top surface 200a.

Instructions 202 are provided on the top surface of 200a, and are permanently imprinted onto the top surface 200a. As contemplated in this embodiment, instructions 202 are listed in sequential order or steps down a first column extending along the width of the handcuff training apparatus 200, each instruction 202 being provided in a separate row of that column. The same instructions 202 are provided in a second column adjacent to the first column, and the same instructions 202 are provided in a third column adjacent to the second column, and so on until the top surface 200a is covered with instructions 202.

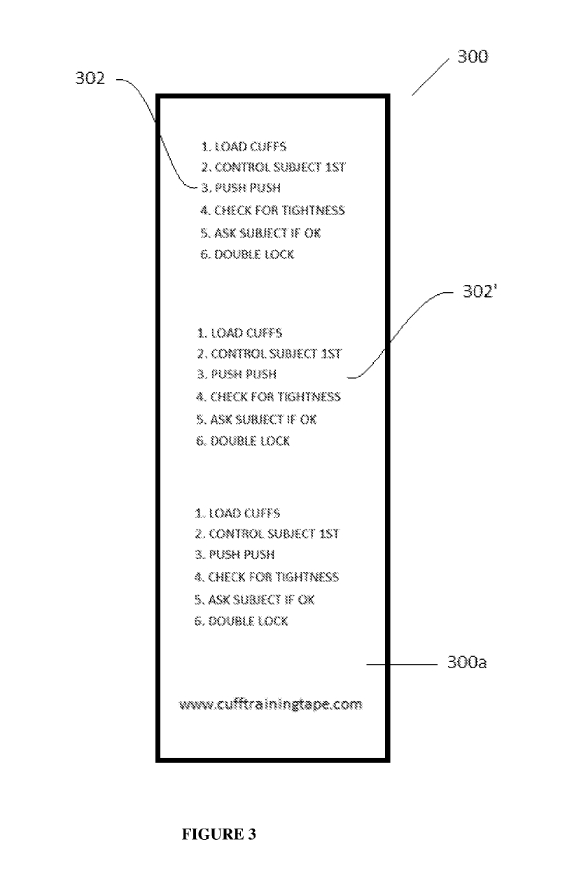

Referring to FIG. 3, and according to another embodiment of the disclosure, there is a handcuff training apparatus 300 adapted for training a first individual on how to apply handcuffs onto the wrists of a second individual. The handcuff training apparatus 300 comprises a top surface 300a and a bottom surface (not shown) that is opposite the top surface 300a. The handcuff training apparatus 300 further comprises instructions (e.g. instructions 302 and 302′) provided on the top surface 300a.

A first set of instructions 302 is provided on the top surface of 300a. As contemplated in this embodiment, the first set of instructions 302 comprises a list of individual steps listed in sequential order down a first column extending along the length of the handcuff training apparatus 300, each step being provided in a separate row of that column. A second set instructions 302′ is provided along the length of the apparatus 300 and below the first set of instructions 302. Sets of instructions are replicated along the length of the apparatus 300 until the top surface 300a is covered with sets of instructions (e.g. instructions 302, 302′).

The handcuff training apparatus 100, 200, 300 is formed of a suitable material that can provide a contouring fit around the second individual's wrist. The material may stretch length-wise (i.e. in the longitudinal direction), width-wise, or both. A non-limiting example of a suitable material having a non-adhesive top surface and an adhesive bottom surface is exercise kinesiology tape.

The handcuff training apparatus 100, 200, 300 has a range of suitable dimensions. The handcuff training apparatus 100, 200, 300 when wrapped around the second individual's wrist, has a thickness that provides protection against contact with a handcuff, but does not interfere with the application of the handcuff around the wrist of the second individual (i.e. the handcuff training apparatus is not too thick so as to impede the application of the handcuff around the wrist of the second individual). The handcuff training apparatus 100, 200, 300 may have a thickness of less than 10 millimetres, less than 5 millimetres, less than 2 millimetres, greater than 1 millimetre, greater than 5 centimetres. For example, the thickness of the handcuff training apparatus 100, 200, 300 can be about 1 to about 2 millimetres.

The handcuff training apparatus 100, 200, 300 has a width that protects at least the second individual's wrist, or a part thereof, and provides a suitable area for the first individual to apply the handcuffs onto the second individual. The handcuff training apparatus 100, 200, 300 may have a width of less than 10 centimetres, less than 5 centimetres, less than 2 centimetres, greater than 1 centimetres, greater than 5 centimetres. For example, the width of the handcuff training apparatus 100, 200, 300 can be about 1, 1.5, 2, 2.5, 3, 3.5, 4, 4.5, 5, 5.5, 6, 6.5, 7, 7.5, 8, 8.5, 9, 9.5, or 10 centimetres.

The handcuff training apparatus 100, 200, 300 has a length (or in other embodiments a circumference) that allows the handcuff training apparatus 100, 200, 300 to circumscribe the wrist of the second individual. The handcuff training apparatus 100, 200, 300 may have a length (or in other embodiments a circumference) ranging between about 12 to about 30 centimetres. For example, the length (or in other embodiments the circumference) of the handcuff training apparatus 100, 200, 300 can be about 12, 13, 14, 15, 16, 17, 18, 19, 20, 21, 22, 23, 24, 25, 26, 27, 28, 29, or 30 centimetres. In a non-limiting example, handcuff training apparatus 100, 200, 300 has a 5 cm width and a 25 cm length.

In other embodiments, the handcuff training apparatus can have a top surface that is non-permanent marking, thereby allowing instructions and/or guiding lines to be provided, modified, or added as desired.

### Method of Using Handcuff Training Apparatus

The present disclosure also relates to a method of using a handcuff training apparatus. The handcuff training apparatus can be used as a tool for training a first individual on how to apply handcuffs onto a second individual in a manner that preferably minimizes damage and/or pain to the second individual.

Using the handcuff training apparatus 100 as an example, the handcuff training apparatus 100 is first wrapped around the second individual's wrist or surrounding area. Following the directions provided by the instructions 102 provided on the top surface 100a of the handcuff training apparatus 100, the first individual executes one or more procedural actions that may occur prior to applying handcuffs onto the second individual. Using the guiding lines 104 as reference, the first individual applies the handcuffs onto the second individual. For example, the first individual can apply the handcuffs onto the second individual along the guiding lines 104 or parallel to the guiding lines 104. Following the directions provided by the instructions 102 provided on the top surface 100a of the handcuff training apparatus 100, the first individual can further execute one or more procedural actions that may occur after applying handcuffs onto the second individual.

### Handcuff Training Apparatus Kit

The present disclosure also relates to a kit comprising a handcuff training apparatus (e.g. handcuff training apparatus 100) and separate instructions on how to use the handcuff training apparatus. The kit can further comprise a pair of handcuffs.

The separate instructions on how to use the handcuff training apparatus can be provided in written or audio form.

It is contemplated that any part of any aspect or embodiment discussed in this specification can be implemented or combined with any part of any other aspect or embodiment discussed in this specification. While particular embodiments have been described in the foregoing, it is to be understood that other embodiments are possible and are intended to be included herein. It will be clear to any person skilled in the art that modification of and adjustment to the foregoing embodiments, not shown, is possible.

1. A handcuff training apparatus comprising a first surface and a second surface that is opposite the first surface, the first surface comprising one or more instructions provided thereon and/or one or more guiding lines extending along a length of the handcuff training apparatus, the handcuff training apparatus adapted to circumscribe and removably couple to a wrist of an individual.

2. The handcuff training apparatus of claim 1, wherein the first surface comprises the one or more instructions and the one or more guiding lines extending along the length of the handcuff training apparatus.

3. The handcuff training apparatus of claim 1, wherein the one or more guiding lines extend along or about a centre-line of the handcuff training apparatus.

4. The handcuff training apparatus of claim 1, wherein the second surface comprises an adhesive adapted to removably couple the handcuff training apparatus to the wrist of the individual.

5. The handcuff training apparatus of claim 1, wherein the handcuff training apparatus stretches in a longitudinal direction.

6. The handcuff training apparatus of claim 5, wherein the handcuff training apparatus is made of kinesiology tape.

7. A method of using the handcuff training apparatus of claim 1, the method comprising:
(a) providing the handcuff training apparatus;
(b) wrapping the handcuff training apparatus around a wrist of an individual; and
(c) applying a handcuff onto the wrist of the individual by:
(i) following the one or more instructions provided on the surface of the handcuff training apparatus; and/or
(ii) using the guiding lines extending along the length of the handcuff training apparatus as a reference.

8. The method of claim 7, further comprising following instructions provided on the first surface of the handcuff training apparatus prior to or subsequent to apply the handcuff onto the wrist of the individual.

9. A handcuff training kit comprising: (i) the handcuff training apparatus of claim 1; and (ii) instructions on how to use the handcuff training apparatus of claim 1.

10. The handcuff training kit of claim 9, wherein the instructions are written or audio.

11. The handcuff training kit of claim 9, further comprising a pair of handcuffs.

12. Use of the handcuff training apparatus of claim 1 for training a first individual on how to apply handcuffs onto a second individual in a manner that minimizes discomfort for the second individual.

13. The handcuff training apparatus of claim 1 for use in training a first individual on how to apply handcuffs onto a second individual in a manner that minimizes discomfort for the second individual.
